(19) United States
(12) Patent Application Publication
(10) Pub. No.: US 2011/0171138 A1
(43) Pub. Date: Jul. 14, 2011

(54) SUBSTANTIALLY PURE DEFERASIROX AND PROCESSES FOR THE PREPARATION THEREOF

(75) Inventors: Rajendra Suryabhan Patil, Tal-Skahapur (IN); Kishore Charugundla, Andhra Pradesh (IN); Praveen Kumar Neela, Andhra Pradesh (IN); Nitin Sharadchandra Pradhan, Maharashtra (IN); Jon Valgeirsson, Hafnarfjordur (IS)

(73) Assignee: ACTAVIS GROUP PTC EHF, Hafnarfjordur (IS)

(21) Appl. No.: 12/995,795

(22) PCT Filed: Jun. 2, 2009
(86) PCT No.: PCT/IB2009/006017

§ 371 Date: Mar. 24, 2011

(30) Foreign Application Priority Data
Jun. 2, 2008 (IN) 1341/CHE/2008

(51) Int. Cl. A61K 31/4196; C07D 249/08; A61K 9/12; A61P 7/00

(52) U.S. Cl. 424/43; 548/269.4; 514/383

(57) ABSTRACT

Provided herein is a highly pure deferasirox or a pharmaceutically acceptable salt thereof substantially free of hydrazine impurity, 4-hydrazinobenzoic acid, processes for the preparation thereof, and pharmaceutical compositions comprising highly pure deferasirox or a pharmaceutically acceptable salt thereof substantially free of hydrazine impurity.

### CROSS REFERENCE TO RELATED APPLICATION

This application claims the benefit of priority to Indian provisional application No. 1341/CHE/2008, filed on Jun. 2, 2008, which is incorporated herein by reference in its entirety.

### FIELD OF THE DISCLOSURE

Disclosed herein is a highly pure deferasirox or a pharmaceutically acceptable salt thereof substantially free of hydrazine impurity, 4-hydrazinobenzoic acid, processes for the preparation thereof, and pharmaceutical compositions comprising highly pure deferasirox or a pharmaceutically acceptable salt thereof substantially free of hydrazine impurity.

### BACKGROUND

U.S. Pat. No. 6,465,504 B1 discloses a variety of substituted 3,5-diphenyl-1,2,4-triazoles, processes for their preparation, pharmaceutical compositions in which they are present, and method of use thereof. These compounds are active as iron chelators and useful in the treatment of iron overload in warm-blooded animals. Among them, deferasirox, 4-[3,5-Bis(2-hydroxyphenyl)-1H-1,2,4-triazol-1-yl]benzoic acid, is an iron chelating agent and it is indicated for the treatment of chronic iron overload due to blood transfusions (transfusional hemosiderosis). Deferasirox is represented by the following structural formula:

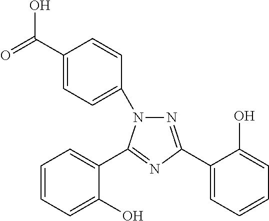

Deferasirox is sold by Novartis under the brand name EXJADE®. Methods of preparing deferasirox are described in U.S. Pat. No. 6,465,504 B1 (herein after referred to as the '504 patent).

The '504 patent describes several synthetic routes for preparing deferasirox. According to one synthetic process, deferasirox is prepared by the ring rearrangement reaction of 1,2,4-dithiazolidine compound with a substituted hydrazine compound in a polar solvent at ambient temperature or elevated temperature up to the reflux temperature of the reaction mixture. However, the experimental details are not provided for this synthetic route.

According to a second synthetic process as described in the '504 patent, deferasirox is prepared by the reaction of 2-(2-hydroxyphenyl)benz[e][1,3]oxazin-4-one with 4-hydrazinobenzoic acid in ethanol at reflux temperature for 2 hours, followed by cooling to precipitate the crystals, and washing with ethanol and then drying to produce deferasirox.

According to a third synthetic process as described in the '504 patent, deferasirox is prepared by the reaction of diacylamine compound with a substituted hydrazine compound in the presence of polar, protic solvents under weak acid catalysis, preferably in aqueous acetic acid at elevated temperature. However, the experimental details are not provided for this synthetic route.

PCT publication No. WO 2008/065123 discloses six crystalline forms, including two solvate forms (forms A, B, C, D, S_{A} & S_{B}), and an amorphous form, of 4-[3,5-bis(2-hydroxyphenyl)-[1,2,4]triazol-1-yl]benzoic acid (deferasirox), and processes for the preparation of the crystalline forms. The crystalline forms are characterized by powder X-ray diffraction (P—XRD), Raman Spectrum and melting points.

U.S. Patent Application No. 2008/0262060 discloses four crystalline forms of deferasirox, methods for the preparation, and pharmaceutical compositions thereof.

It is known that synthetic compounds can contain extraneous compounds or impurities resulting from their synthesis or degradation. The impurities can be unreacted starting materials, by-products of the reaction, products of side reactions, or degradation products. Generally, impurities in an active pharmaceutical ingredient (API) may arise from degradation of the API itself, or during the preparation of the API. Impurities in deferasirox or any active pharmaceutical ingredient (API) are undesirable and might be harmful.

Regulatory authorities worldwide require that drug manufactures isolate, identify and characterize the impurities in their products. Furthermore, it is required to control the levels of these impurities in the final drug compound obtained by the manufacturing process and to ensure that the impurity is present in the lowest possible levels, even if structural determination is not possible.

The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the active pharmaceutical ingredient, the product must be analyzed for purity, typically, by HPLC, TLC or GC analysis, to determine if it is suitable for continued processing and, ultimately, for use in a pharmaceutical product. Purity standards are set with the intention of ensuring that an API is as free of impurities as possible, and, thus, are as safe as possible for clinical use. The United States Food and Drug Administration guidelines recommend that the amounts of some impurities limited to less than 0.1 percent.

Generally, impurities are identified spectroscopically and by other physical methods, and then the impurities are associated with a peak position in a chromatogram (or a spot on a TLC plate). Thereafter, the impurity can be identified by its position in the chromatogram, which is conventionally measured in minutes between injection of the sample on the column and elution of the particular component through the detector, known as the “retention time” (“Rt”). This time period varies daily based upon the condition of the instrumentation and many other factors. To mitigate the effect that such variations have upon accurate identification of an impurity, practitioners use “relative retention time” (“RRt”) to identify impurities. The RRt of an impurity is its retention time divided by the retention time of a reference marker.

Accordingly, there remains a need for highly pure deferasirox substantially free of impurities, as well as purification processes for obtaining thereof.

### SUMMARY

In one aspect, provided herein is a highly pure deferasirox or a pharmaceutically acceptable salt thereof substantially free of hydrazine impurity, 4-hydrazinobenzoic acid.

As used herein “highly pure deferasirox or a pharmaceutically acceptable salt thereof substantially free of hydrazine impurity” refers to deferasirox or a pharmaceutically acceptable salt thereof, in which deferasirox has a purity of about 99% to about 99.99% and further comprising hydrazine impurity in an amount of less than about 50 ppm (parts per million). Specifically, the deferasirox, as disclosed herein, contains less than about 5 ppm, more specifically less than about 1 ppm, still more specifically less than about 0.5 ppm of hydrazine impurity, and most specifically is essentially free of hydrazine impurity.

The content of hydrazine impurity can be measured by the analytical techniques such as High performance liquid chromatography (HPLC) and Liquid chromatography-mass spectrometry (LC-MS). The content of hydrazine impurity is preferably measured by HPLC. The term “deferasirox or a pharmaceutically acceptable salt thereof essentially free of hydrazine impurity” means deferasirox or a pharmaceutically acceptable salt thereof contains a non-detectable amount of hydrazine impurity as measured by HPLC.

In another aspect, provided herein is deferasirox or a pharmaceutically acceptable salt thereof that comprises hydrazine impurity in an amount of less than about 1 ppm, and specifically in an amount of less than about 0.5 ppm, as measured by HPLC.

In another aspect, provided herein is deferasirox or a pharmaceutically acceptable salt thereof essentially free of the hydrazine impurity.

In another aspect, provided herein is deferasirox or a pharmaceutically acceptable salt thereof having a purity of greater than about 99%, specifically greater than about 99.9%, and more specifically greater than about 99.95%, as measured by HPLC.

In still further aspect, encompassed herein is a process for preparing the highly pure deferasirox or a pharmaceutically acceptable salt thereof substantially free of hydrazine impurity.

In another aspect, provided herein is a pharmaceutical composition comprising highly pure deferasirox or a pharmaceutically acceptable salt thereof substantially free of hydrazine impurity, and one or more pharmaceutically acceptable excipients.

In still another aspect, provided herein is a pharmaceutical composition comprising highly pure deferasirox or a pharmaceutically acceptable salt thereof substantially free of hydrazine impurity made by the process disclosed herein, and one or more pharmaceutically acceptable excipients.

In still further aspect, encompassed is a process for preparing a pharmaceutical formulation comprising combining highly pure deferasirox or a pharmaceutically acceptable salt thereof substantially free of hydrazine impurity with one or more pharmaceutically acceptable excipients.

In another aspect, the highly pure deferasirox or a pharmaceutically acceptable salt thereof substantially free of hydrazine impurity disclosed herein for use in the pharmaceutical compositions has a 90 volume-percent of the particles (D₉₀) having a size of less than or equal to about 400 microns, specifically less than or equal to about 300 microns, more specifically less than or equal to about 100 microns, still more specifically less than or equal to about 60 microns, and most specifically less than or equal to about 15 microns.

### DETAILED DESCRIPTION

Deferasirox obtained by the processes described in the art is not satisfactory from a purity perspective. Reproduction of the deferasirox synthetic procedures as described in the prior art show that unacceptable amounts of impurities are generally formed along with deferasirox. Among these impurities, 4-hydrazinobenzoic acid (hereinafter referred to as the ‘hydrazine impurity’) of formula I:

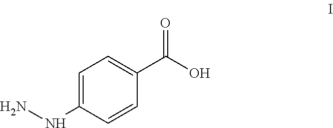

is the main concern. 4-hydrazinobenzoic acid, which has a genotoxic and carcinogenic potential, has been identified and isolated. In a specific experiment, the inventors have found that deferasirox prepared by the prior art procedures contained about above 50 ppm (parts per million) and up to 100 ppm of the hydrazine impurity.

Extensive experimentation has been carried out by the present inventors to reduce the level of the hydrazine impurity in deferasirox. As a result, it has been found that the hydrazine impurity formed in the preparation of the deferasirox can be reduced in two ways as follows: i) providing a solution of crude deferasirox in a solvent selected from the group consisting alcohols, ketones and mixtures thereof, partially removing the solvent from the solution, and isolating pure deferasirox substantially free of hydrazine impurity from the solution by using a suitable isolation technique; ii) dissolving the crude deferasirox in an aqueous alkali solution, adding a co-solvent to the solution, adjusting the pH of the solution to below about 2.5 using a suitable acid, and recovering the pure deferasirox substantially free of hydrazine impurity.

In addition to the presence of impurities, the solid state physical properties of an active pharmaceutical ingredient (API), such as deferasirox, can be very important in formulating a drug substance, and can have profound effects on the ease and reproducibility of formulation. Particle size, for example, may affect the flowability and mixability of a drug substance. In cases where the active ingredient has good flow properties, tablets can be prepared by direct compression of the ingredients. However, in many cases, the particle size of the active substance is very small, the active substance is cohesive, or the active substance has poor flow properties. Small particles are also filtered and washed more slowly during isolation processes, and thus may increase the time and expense of manufacturing a drug formulation.

Deferasirox is a white to slightly yellow non-hygroscopic powder and it is practically insoluble in water and in acid medium, the solubility increasing with pH. The lack of solubility of deferasirox is problematic since bioavailability of a water insoluble active ingredient is usually poor. There is a need to prepare active pharmaceutical ingredients such as deferasirox particles with a desired surface area to obtain formulations with greater bioavailability, and to compensate for any loss of surface area before formulation.

According to one aspect, provided herein is a highly pure deferasirox or a pharmaceutically acceptable salt thereof, substantially free of hydrazine impurity, 4-hydrazinobenzoic acid.

As used herein “highly pure deferasirox or a pharmaceutically acceptable salt thereof substantially free of hydrazine impurity” refers to deferasirox or a pharmaceutically acceptable salt thereof, in which deferasirox has a purity of about 99% to about 99.99% and further comprising hydrazine impurity in an amount of less than about 50 ppm (parts per million) as measured by HPLC. Specifically, the highly pure deferasirox, as disclosed herein, contains less than about 5 ppm, more specifically less than about 1 ppm, still more specifically less than about 0.5 ppm of hydrazine impurity, and most specifically is essentially free of hydrazine impurity.

The term “deferasirox or a pharmaceutically acceptable salt thereof essentially free of hydrazine impurity” means deferasirox or a pharmaceutically acceptable salt thereof contains a non-detectable amount of hydrazine impurity as measured by HPLC.

According to another aspect, there is provided a process for the purification of deferasirox, comprising:
a) providing a solution of crude deferasirox in a solvent selected from the group consisting of an alcohol, a ketone, and mixtures thereof;
b) optionally, subjecting the solution obtained in step-(a) to carbon treatment or silica gel treatment;
c) partially removing the solvent from the solution; and
d) precipitating pure deferasirox substantially free of hydrazine impurity from the solution.

The term ‘crude deferasirox’ in the specification refers to deferasirox containing the hydrazine impurity in an amount of greater than about 50 ppm.

Exemplary alcohol solvents used in step-(a) include, but are not limited to, C₁ to C₈ straight or branched chain alcohol solvents such as methanol, ethanol, n-propanol, isopropanol, n-butanol, isobutanol, tert-butanol, amyl alcohol, isoamyl alcohol, hexanol, and mixtures thereof. A specific alcohol solvent is methanol.

Exemplary ketone solvents used in step-(a) include, but are not limited to, acetone, methyl ethyl ketone, methyl isobutyl ketone, methyl tert-butyl ketone and the like, and mixtures thereof. A specific ketone solvent is acetone.

Step-(a) of providing a solution of crude deferasirox includes dissolving crude deferasirox in the solvent, or obtaining an existing solution from a previous processing step.

In one embodiment, the crude deferasirox is dissolved in the solvent at a temperature of above about 25° C., specifically at about 25° C. to about 100° C., and more specifically at about 40° C. to about 80° C.

In another embodiment, the solution in step-(a) is prepared by reacting 2-(2-hydroxyphenyl)-4H-1,3-benzoxazin-4-one with 4-hydrazinobenzoic acid in a reaction inert solvent under suitable conditions to produce a reaction mass containing crude deferasirox, followed by usual work up such as washings, extractions, evaporations, etc. In one embodiment, the work-up includes dissolving or extracting the resulting deferasirox in the solvent at a temperature of above about 25° C., specifically at about 25° C. to about 100° C., and more specifically at about 40° C. to about 80° C.

Exemplary reaction inert solvents suitable for facilitating the reaction between 2-(2-hydroxyphenyl)-4H-1,3-benzoxazin-4-one and 4-hydrazinobenzoic acid include, but are not limited to, water, alcohols, ketones, cyclic ethers, aliphatic ethers, hydrocarbons, chlorinated hydrocarbons, nitriles, esters, polar aprotic solvents, and mixtures thereof. In one embodiment, the solvent is selected from the group consisting of water, methanol, ethanol, n-propanol, isopropanol, n-butanol, isobutanol, tert-butanol, amyl alcohol, hexanol, acetone, and mixtures thereof. A specific reaction inert solvent is ethanol.

The solution obtained in step-(a) is optionally stirred at a temperature of above about 25° C. for at least 15 minutes, and specifically at a temperature of about 40° C. to about 80° C. for about 20 minutes to about 5 hours.

The carbon treatment or silica gel treatment in step-(b) is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate containing deferasirox by removing charcoal or silica gel. Specifically, the finely powdered carbon is an active carbon. A specific mesh size of silica gel is 40-500 mesh, and more specifically 60-120 mesh.

The term “partially removing” the solvent in step-(c) refers to at least 30% by volume, specifically about 35% to about 80% by volume, more specifically about 40% to about 60% by volume, still more specifically about 50% by volume, removal of the solvent from the solvent solution.

Removal of solvent in step-(c) is accomplished, for example, by partial evaporation of the solvent, concentrating the solution or distillation of solvent under inert atmosphere, or a combination thereof, to substantial elimination of total solvent present in the reaction mass.

The distillation process can be performed at atmospheric pressure or reduced pressure. Specifically, the distillation is carried out at a temperature of about 30° C. to about 110° C., more specifically at about 40° C. to about 90° C., and most specifically at about 45° C. to about 80° C.

Specifically, the solvent is removed at a pressure of about 760 mm Hg or less, more specifically at about 400 mm Hg or less, still more specifically at about 80 mm Hg or less, and most specifically from about 30 to about 80 mm Hg.

The precipitation of pure deferasirox substantially free of hydrazine impurity in step-(d) is carried out by cooling the solution at a temperature of below 25° C. for at least 15 minutes, specifically at about 0° C. to about 25° C. for about 30 minutes to about 20 hours, and more specifically at about 0° C. to about 20° C. for about 1 hour to about 4 hours.

The pure deferasirox substantially free of hydrazine impurity obtained in step-(d) is recovered by techniques such as filtration, filtration under vacuum, decantation, centrifugation, or a combination thereof. In one embodiment, the deferasirox is recovered by filtration employing a filtration media of, for example, a silica gel or celite.

The highly pure deferasirox or a pharmaceutically acceptable salt thereof substantially free of hydrazine impurity obtained by the above process may be further dried in, for example, a Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In one embodiment, the drying is carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer, and the like. Drying equipment selection is well within the ordinary skill in the art.

According to another aspect, there is provided a process for purifying deferasirox, comprising:
a) suspending crude deferasirox in water to provide a suspension;
b) combining the suspension with an aqueous alkali metal hydroxide solution to form a first reaction mixture;
c) heating the first reaction mixture obtained in step-(b) to form a clear solution;
d) admixing the clear solution with a co-solvent selected from the group consisting of water, an alcohol, a ketone, and mixtures thereof, to form a second reaction mixture; and
e) precipitating pure deferasirox substantially free of hydrazine impurity by adjusting the pH of the second reaction mixture obtained in step-(d) to pH 1 to 3 with an acid.

Step-(a) of providing a suspension of deferasirox includes suspending crude deferasirox in water while stirring. In one embodiment, the suspension is stirred for at least 15 minutes at a temperature of below about 90° C., and more specifically for about 30 minutes to about 3 hours at about 0° C. to about 50° C.

In another embodiment, the suspension in step-(a) is prepared by reacting 242-hydroxyphenyl)-4H-1,3-benzoxazin-4-one with 4-hydrazinobenzoic acid as described above.

In one embodiment, the alkali metal hydroxide used in step-(b) is sodium hydroxide or potassium hydroxide. A specific alkali metal hydroxide is sodium hydroxide.

Combining of the suspension with aqueous alkali metal hydroxide solution in step-(b) is done in a suitable order, for example, the suspension is added to the aqueous alkali metal hydroxide solution, or alternatively, the aqueous alkali metal hydroxide solution is added to the suspension. The addition is, for example, carried out drop wise, in one portion, or in more than one portion. The addition is specifically carried out drop wise at a temperature of below 50° C. for at least 20 minutes, and more specifically at a temperature of about 15° C. to about 35° C. for about 30 minutes to about 2 hours.

The heating in step-(c) is carried out at a temperature of about 40° C. to about 80° C. for at least 20 minutes, and more specifically at a temperature of about 50° C. to about 75° C. for about 30 minutes to about 4 hours. After completion of the heating step, the solution is specifically cooled at a temperature of below about 35° C. for at least 15 minutes, and more specifically at about 0° C. to about 30° C. for about 30 minutes to about 3 hours.

The solution obtained in step-(c) is optionally subjected to carbon treatment or silica gel treatment. The carbon treatment or silica gel treatment is carried out by methods known in the art, for example, by stirring the solution with finely powdered carbon or silica gel at a temperature of below about 70° C. for at least 15 minutes, specifically at a temperature of about 40° C. to about 70° C. for at least 30 minutes; and filtering the resulting mixture through hyflo to obtain a filtrate by removing charcoal or silica gel. Specifically, the finely powdered carbon is an active carbon. A specific mesh size of silica gel is 40-500 mesh, and more specifically 60-120 mesh.

Exemplary alcohol solvents used in step-(d) include, but are not limited to, C₁ to C₈ straight or branched chain alcohol solvents such as methanol, ethanol, n-propanol, isopropanol, n-butanol, isobutanol, tert-butanol, amyl alcohol, isoamyl alcohol, hexanol, and mixtures thereof. Specific alcohol solvents are methanol, isopropanol, and mixtures thereof.

Exemplary ketone solvents used in step-(d) include, but are not limited to, acetone, methyl ethyl ketone, methyl isobutyl ketone, methyl tert-butyl ketone and the like, and mixtures thereof. A specific ketone solvent is acetone.

The admixing in step-(d) is done in a suitable order, for example, the solution is added to the co-solvent, or alternatively, the co-solvent is added to the solution. The addition is, for example, carried out drop wise, in one portion, or in more than one portion. The addition is specifically carried out at a temperature of below 50° C. for at least 15 minutes, and more specifically at a temperature of about 15° C. to about 35° C. for about 20 minutes to about 2 hours while stirring.

In one embodiment, the pH of the second reaction mixture in step-(e) is adjusted to 1.0 to 2.5, and more specifically to 1.5 to 2.5.

The acid used in step-(e) is an organic acid or a mineral acid. Exemplary organic acids are acetic acid and formic acid.

In one embodiment, the acid used in step-(e) is a mineral acid such as sulfuric acid, hydrochloric acid, hydrobromic acid, hydroiodic acid and phosphoric acid. A specific mineral acid is hydrochloric acid.

In another embodiment, the hydrochloric acid used is in the form of concentrated hydrochloric acid or aqueous hydrochloric acid or in the form of hydrogen chloride gas or hydrogen chloride dissolved in an organic solvent. The organic solvent used for dissolving hydrogen chloride gas or hydrogen chloride is selected from the group consisting of ethanol, methanol, isopropyl alcohol, ethyl acetate, and acetone.

The highly pure deferasirox substantially free of hydrazine impurity obtained in step-(e) is recovered and further dried by the methods described hereinabove.

If required, pure deferasirox substantially free of hydrazine impurity obtained by the processes described above may be converted into pharmaceutically acceptable salts by conventional methods.

Pharmaceutically acceptable salts of deferasirox can be prepared in high purity by using the pure deferasirox substantially free of hydrazine impurity obtained by the method disclosed herein, by known methods.

In one embodiment, the pharmaceutically acceptable salts of deferasirox are obtained from alkali or alkaline earth metals include the sodium, calcium, potassium and magnesium; transition metal salts such as zinc salts; organic amines such as ethylamine, tert-butylamine, diethylamine, and diisopropylamine.

Exemplary pharmaceutically acceptable salts of deferasirox include, but are not limited to, triethylamine salt, dimethylamine salt, tert-butylamine salt, sodium (Na⁺) salt, potassium (K⁺) salt, magnesium (Mg²⁺) salt, calcium (Ca²⁺) salt and zinc (Zn²⁺) salt.

According to another aspect, the highly pure deferasirox or a pharmaceutically acceptable salt thereof substantially free of hydrazine impurity obtained by the process disclosed herein has a relatively low content of one or more organic volatile impurities

In one embodiment, the deferasirox or a pharmaceutically acceptable salt thereof substantially free of hydrazine impurity obtained by the processes disclosed herein comprises less than about 2000 parts per million (ppm) methanol, less than about 200 ppm tetrahydrofuran, less than about 200 ppm toluene, and less than about 200 ppm dimethylformamide, as measured by Gas Chromatography (GC). Specifically, the deferasirox or a pharmaceutically acceptable salt thereof substantially free of hydrazine impurity obtained by the process disclosed herein comprises less than about 1500 ppm methanol, less than about 10 ppm tetrahydrofuran, less than about 10 ppm toluene, and less than about 10 ppm dimethylformamide.

In another embodiment, deferasirox or a pharmaceutically acceptable salt thereof substantially free of hydrazine impurity obtained by the processes disclosed herein has the overall level of organic volatile impurities less than about 1500 ppm, and more specifically less than about 1400 ppm.

Further encompassed herein is the use of the highly pure deferasirox or a pharmaceutically acceptable salt thereof substantially free of hydrazine impurity for the manufacture of a pharmaceutical composition together with a pharmaceutically acceptable carrier.

A specific pharmaceutical composition of highly pure deferasirox or a pharmaceutically acceptable salt thereof substantially free of hydrazine impurity is selected from a solid dosage form and an oral suspension.

In one embodiment, the highly pure deferasirox or a pharmaceutically acceptable salt thereof substantially free of hydrazine impurity has a D₉₀ particle size of less than or equal to about 400 microns, specifically less than or equal to about 300 microns, more specifically less than or equal to about 100 microns, still more specifically less than or equal to about 60 microns, and most specifically less than or equal to about 15 microns.

In another embodiment, the particle sizes of the highly pure deferasirox or a pharmaceutically acceptable salt thereof substantially free of hydrazine impurity are produced by a mechanical process of reducing the size of particles which includes any one or more of cutting, chipping, crushing, milling, grinding, micronizing, trituration or other particle size reduction methods known in the art, to bring the solid state form to the desired particle size range.

According to another aspect, there is provided a method for treating a patient suffering from chronic iron overload, comprising administering a therapeutically effective amount of the highly pure deferasirox or a pharmaceutically acceptable salt thereof substantially free of hydrazine impurity, or a pharmaceutical composition that comprises a therapeutically effective amount of highly pure deferasirox or a pharmaceutically acceptable salt thereof substantially free of hydrazine impurity, along with pharmaceutically acceptable excipients.

According to another aspect, there is provided pharmaceutical compositions comprising highly pure deferasirox or a pharmaceutically acceptable salt thereof substantially free of hydrazine impurity prepared according to processes disclosed herein and one or more pharmaceutically acceptable excipients.

According to another aspect, there is provided a process for preparing a pharmaceutical formulation comprising combining highly pure deferasirox or a pharmaceutically acceptable salt thereof substantially free of hydrazine impurity prepared according to processes disclosed herein, with one or more pharmaceutically acceptable excipients.

Yet in another embodiment, pharmaceutical compositions comprise at least a therapeutically effective amount of highly pure deferasirox or a pharmaceutically acceptable salt thereof substantially free of hydrazine impurity. Such pharmaceutical compositions may be administered to a mammalian patient in a dosage form, e.g., solid, liquid, powder, elixir, aerosol, syrups, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, syrup, troches, sachets, suspensions, powders, lozenges, elixirs and the like. The highly pure deferasirox or a pharmaceutically acceptable salt thereof substantially free of hydrazine impurity may also be administered as suppositories, ophthalmic ointments and suspensions, and parenteral suspensions, which are administered by other routes.

The dosage forms may contain highly pure deferasirox or a pharmaceutically acceptable salt thereof substantially free of hydrazine impurity as is or, alternatively, may contain highly pure deferasirox or a pharmaceutically acceptable salt thereof substantially free of hydrazine impurity as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In one embodiment, capsule dosage forms contain highly pure deferasirox or a pharmaceutically acceptable salt thereof substantially free of hydrazine impurity within a capsule which may be coated with gelatin. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, the coating agents may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions described herein may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols such as mannitol and sorbitol, acrylate polymers and copolymers, as well as pectin, dextrin and gelatin.

Other excipients include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

The following examples are given for the purpose of illustrating the present disclosure and should not be considered as limitation on the scope or spirit of the disclosure.

### Experimental:

### High Performance Liquid Chromatography (HPLC):

The content of 4-hydrazinobenzoic acid impurity in deferasirox was measured by high performance liquid chromatography by using Water's HPLC system having alliance 2695 model pump and 2487 (UV) detector with Empower chromatography software or its equivalent under the following conditions:
Column: ACE 3 C18, (100×4.6 mm, 3.0 μm) Make: ACE, Catalogue No: ACE-111-1046
Column filter: 2.0μ pre filter, Make: Alltech, Part No: 28689
Detector: UV at 260 nm
Flow rate: 0.70 ml/min
Injection volume: 75.0 μL
Run time: 45 min
Oven temperature: 35° C.
Diluent: water
Elution: Gradient

### Buffer Preparation:

About 10.0 ml of acetic acid was transferred into 1000 ml of water followed by filtration through 0.22μ porosity membrane and degassed.

### Mobile Phase-A: Buffer (100%).

Mobile Phase-B: Buffer and Acetonitrile (40:60% v/v).

### REFERENCE EXAMPLE

### Preparation of Deferasirox (Crude)

2-(2-Hydroxyphenyl)-4H-1,3-benzoxazin-4-one (25 g, 0.1045 mol), 4-hydrazinobenzoic acid (17.5 g, 0.115 mol) and methanol (375 ml) were added to a round bottom flask. The reaction mixture was heated to reflux temperature and refluxed for 2 hours. The resulting mass was cooled to 25° C. and then filtered to isolate the solid product. The wet material was added to methanol (1625 ml) and heated to 65 to 66° C. to provide a clear solution. The resulting solution mass was cooled to 25° C., and the separated solid was filtered and then dried to produce 28.5 g of crude deferasirox (HPLC Purity: 99.8%, content of 4-hydrazinobenzoic acid impurity: 60 ppm).

### EXAMPLES

### Example 1

Crude deferasirox (50 g, obtained in the above reference example) was added to methanol (2500 ml) and heated at 60-65° C. to provide a clear solution. The resulting clear solution was treated with activated carbon and filtered through hyflow bed. The solution was concentrated to 1250 ml, cooled at 20 to 25° C., and stirred for 1 hour. The precipitated solid was filtered and washed with methanol (150 ml) and then dried to produce 40 g of pure deferasirox (HPLC purity: 99.92%; content of 4-hydrazinobenzoic acid impurity: 0.6 ppm).

Level of organic volatile impurities: methanol: 1395 ppm.

### Example 2

Crude deferasirox (50 g, obtained in the above reference example) was added to acetone (1250 ml) and heated at reflux to provide a clear solution. The resulting solution was treated with activated carbon and filtered through hyflow bed. The solution was concentrated to 500 ml, cooled at 20 to 25° C. and stirred for 1 hour. The precipitated solid was filtered and washed with chilled acetone (100 ml) and then dried to produce 38 g of pure deferasirox (HPLC purity: 99.93%; content of 4-hydrazinobenzoic acid impurity: 1.02 ppm).

### Example 3

Crude deferasirox (3.33 g, 8.919 mmol, obtained in the above reference example) was suspended in water (17 ml) at 22 to 25° C., and then a solution of sodium hydroxide (0.392 g, 9.8 mmol) in water (5.0 ml) was added drop wise to it. The mixture was heated to 55° C. to provide a clear solution, followed by cooling to 25° C. Acetone (25 ml) was added to above solution and pH was adjusted to 2.0 to 2.5 with a concentrated hydrochloric acid solution (1.1 ml) at 22 to 25° C. The solid precipitate was further stirred for 30 minutes. The precipitated solid was filtered and dried to produce 2.9 g of pure deferasirox (HPLC purity: 99.99%; content of 4-hydrazinobenzoic acid impurity: not detected).

### Example 4

Crude deferasirox (3.33 g, 8.919 mmol, obtained in the above reference example) was suspended in water (17 ml) at 22 to 25° C. and a solution of sodium hydroxide (0.392 g, 9.8 mmol) in water (5 ml) was added drop wise to it. The mixture was heated to 55° C. to provide a clear solution, followed by cooling to 25° C. Isopropyl alcohol (25 ml) was added to the clear solution. The resulting mixture was followed by drop wise addition of concentrated hydrochloric acid (1.1 ml) at 22 to 25° C. The precipitated solid was further stirred for 30 minutes. The resulting solid was filtered and dried to produce 3.1 g of pure deferasirox (HPLC purity: 99.95%; content of 4-hydrazinobenzoic acid impurity: not detected).

### Example 5

Crude deferasirox (3.33 g, 8.919 mmol, obtained in the above reference example) was suspended in water (17 ml) at 22 to 25° C. and a solution of sodium hydroxide (0.392 g, 9.8 mmol) in water (5 ml) was added drop wise to it. The mixture was heated to 55° C. to provide a clear solution, followed by cooling to 25° C. Methanol (25 ml) was added to the clear solution. The resulting mixture was followed by drop wise addition of concentrated hydrochloric acid (1.1 ml) at 22 to 25° C. The precipitated solid was further stirred for 30 minutes. The resulting solid was filtered and dried to produce 3.3 g of pure deferasirox (HPLC purity: 99.99%; content of 4-hydrazinobenzoic acid impurity: not detected).

Level of organic volatile impurities: methanol: 690 ppm.

### Example 6

Crude deferasirox (3.33 g, 8.919 mmol, obtained in the above reference example) was suspended in water (17 ml) at 22 to 25° C. and a solution of sodium hydroxide (0.392 g, 9.8 mmol) in water (5 ml) was added drop wise to it. The mixture was heated to 55° C. to provide a clear solution, followed by cooling to 25° C. Water (25 ml) was added to the clear solution. The resulting mixture was followed by drop wise addition of concentrated hydrochloric acid (1.1 ml) at 22 to 25° C. The precipitated solid was further stirred for 30 minutes. The resulting solid was filtered and dried to produce 2.7 g of pure deferasirox (HPLC purity: 99.95%; content of 4-hydrazinobenzoic acid impurity: 0.6 ppm).

Unless otherwise indicated, the following definitions are set forth to illustrate and define the meaning and scope of the various terms used to describe the invention herein.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions encompass any composition made by admixing the active ingredient, active ingredient dispersion or composite, additional active ingredient(s), and pharmaceutically acceptable excipients.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia, alginic acid, tragacanth, carboxymethylcellulose sodium, polyvinylpyrrolidone, compressible sugar (e.g., NuTab), ethylcellulose, gelatin, liquid glucose, methylcellulose, pregelatinized starch, starch, polyethylene glycol, guar gum, polysaccharide, bentonites, sugars, invert sugars, poloxamers (PLURONIC™ F68, PLURONIC™ F127), collagen, albumin, celluloses in non-aqueous solvents, polypropylene glycol, polyoxyethylene-polypropylene copolymer, polyethylene ester, polyethylene sorbitan ester, polyethylene oxide, microcrystalline cellulose, combinations thereof and other material known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, microcrystalline cellulose (e.g., Avicel™), carsium (e.g., Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type) is another useful wetting agent, combinations thereof and other such materials known to those of ordinary skill in the art.

As used herein, D_{X} means that X percent of the particles have a diameter less than a specified diameter D. Thus, a D₉₀ or d(0.9) of less than 300 microns means that 90 volume-percent of the particles in a composition have a diameter less than 300 microns.

The term “micronization” used herein means a process or method by which the size of a population of particles is reduced.

As used herein, the term “micron” or “μm” both refer to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Deferasirox or a pharmaceutically acceptable salt thereof comprising 4-hydrazinobenzoic acid in an amount of less than about 50 parts per million.

2. The deferasirox of claim 1, having a purity of about 99% to about 99.99% as measured by HPLC and comprising the 4-hydrazinobenzoic acid in an amount of less than about 5 ppm.

3. (canceled)

4. (canceled)

5. The deferasirox of claim 42, comprising the 4-hydrazinobenzoic acid in an amount of less than about 0.5 ppm.

6. (canceled)

7. A purification process for obtaining highly pure deferasirox or a pharmaceutically acceptable salt thereof of claim 1, comprising:
a) providing a solution of crude deferasirox in a solvent selected from the group consisting of an alcohol, a ketone, and mixtures thereof;
b) optionally, subjecting the solution obtained in step-(a) to carbon treatment or silica gel treatment;
c) partially removing the solvent from the solution; and
d) precipitating pure deferasirox substantially free of hydrazine impurity from the solution;
wherein the alcohol solvent is selected from the group consisting of methanol, ethanol, n-propanol, isopropanol, n-butanol, isobutanol, tert-butanol, amyl alcohol, isoamyl alcohol, hexanol, and mixtures thereof; and wherein the ketone solvent is selected from the group consisting of acetone, methyl ethyl ketone, methyl isobutyl ketone, methyl tert-butyl ketone, and mixtures thereof.

8. (canceled)

9. The process of claim 7, wherein the alcohol solvent is methanol; and wherein the ketone solvent is acetone.

10. The process of claim 7, wherein the solution in step-(a) is provided by dissolving the crude deferasirox in the solvent at a temperature of above about 25° C.; wherein the carbon treatment or silica gel treatment in step-(b) is carried out by stirring the solution with finely powdered carbon or silica gel at a temperature of below about 70° C. for at least 15 minutes, and filtering the resulting mixture through a filtration bed to obtain a filtrate containing deferasirox by removing charcoal or silica gel; wherein the removal of solvent in step-(c) is accomplished by partial evaporation of the solvent, concentrating the solution or distillation of solvent under inert atmosphere, or a combination thereof; wherein the precipitation in step-(d) is carried out by cooling the solution at a temperature of below 25° C. for at least 15 minutes; wherein the deferasirox substantially free of hydrazine impurity obtained in step-(d) is recovered by filtration, filtration under vacuum, decantation, centrifugation, filtration employing a filtration media of a silica gel or celite, or a combination thereof; and wherein the deferasirox substantially free of hydrazine impurity obtained in step-(d) is further dried under vacuum or at atmospheric pressure, at a temperature of about 35° C. to about 70° C.

11. The process of claim 10, wherein the crude deferasirox is dissolved in the solvent at a temperature of about 40° C. to about 80° C.; wherein about 35% to about 80% of the solvent in step-(c) is removed from the solvent solution; and wherein the precipitation in step-(d) is carried out by cooling the solution at a temperature of about 0° C. to about 25° C.

12.-21. (canceled)

22. A purification process for obtaining highly pure deferasirox or a pharmaceutically acceptable salt thereof of claim 1, comprising
a) suspending crude deferasirox in water to provide a suspension;
b) combining the suspension with an aqueous alkali metal hydroxide solution to form a first reaction mixture;
c) heating the first reaction mixture obtained in step-(b) to form a clear solution;
d) admixing the clear solution with a co-solvent selected from the group consisting of an alcohol, a ketone, and mixtures thereof to form a second reaction mixture; and
e) precipitating pure deferasirox substantially free of hydrazine impurity by adjusting the pH of the second reaction mixture obtained in step-(d) to pH 1 to 3 with an acid.

23. The process of claim 22, wherein the suspension in step-(a) is provided by suspending crude deferasirox in water while stirring at a temperature of below about 90° C. for at least 15 minutes; wherein the combining in step-(b) is accomplished by adding the suspension to the aqueous alkali metal hydroxide solution or by adding to the aqueous alkali metal hydroxide solution to the suspension; wherein the first reaction mixture in step-(c) is heated at a temperature of about 40° C. to about 80° C. for at least 20 minutes; wherein the heated solution obtained in step-(c) is cooled at a temperature of below about 35° C. for at least 15 minutes; wherein the solution obtained in step-(c) is optionally subjected to carbon treatment or silica gel treatment; wherein the admixing in step-(d) is carried out either by adding the solution to the co-solvent or by adding the co-solvent to the solution; wherein the pH of the reaction mixture in step-(e) is adjusted to 1.0 to 2.5; and wherein the deferasirox substantially free of hydrazine impurity obtained in step-(e) is recovered by filtration, filtration under vacuum, decantation, centrifugation, filtration employing a filtration media of a silica gel or celite, or a combination thereof.

24. The process of claim 23, wherein the suspension is stirred at a temperature of about 0° C. to about 50° C. for about 30 minutes to about 3 hours; wherein the addition in step-(b) is carried out drop wise at a temperature of below 50° C.; wherein the first reaction mixture in step-(c) is heated at a temperature of about 50° C. to about 75° C. for about 30 minutes to about 4 hours; wherein the heated solution in step-(c) is cooled at a temperature of about 0° C. to about 30° C. for about 30 minutes to about 3 hours; wherein the pH of the reaction mixture in step-(e) is adjusted to 1.5 to 2.5; and wherein the deferasirox substantially free of hydrazine impurity obtained in step-(e) is further dried under vacuum or at atmospheric pressure, at a temperature of about 35° C. to about 70° C.

25. The process of claim 22, wherein the alkali metal hydroxide used in step-(b) is sodium hydroxide or potassium hydroxide; wherein the alcohol solvent used in step-(d) is selected from the group consisting of methanol, ethanol, n-propanol, isopropanol, n-butanol, isobutanol, tert-butanol, amyl alcohol, isoamyl alcohol, hexanol, and mixtures thereof; wherein the ketone solvent used in step-(d) is selected from the group consisting of acetone, methyl ethyl ketone, methyl isobutyl ketone, methyl tert-butyl ketone, and mixtures thereof; and wherein the acid used in step-(e) is an organic acid or a mineral acid.

26. The process of claim 25, wherein the alkali metal hydroxide used in step-(b) is sodium hydroxide; wherein the alcohol solvent used in step-(d) is selected from the group consisting of methanol, isopropanol, and mixtures thereof; wherein the ketone solvent used in step-(d) is acetone; wherein the organic acid used in step-(e) is acetic acid or formic acid; and wherein the mineral acid used in step-(e) is sulfuric acid, hydrochloric acid, hydrobromic acid, hydroiodic acid, or phosphoric acid.

27.-49. (canceled)

50. The process of claim 22, wherein the deferasirox or a pharmaceutically acceptable salt thereof substantially free of 4-hydrazinobenzoic acid obtained has less than about 2000 parts per million methanol as measured by Gas Chromatography (GC).

51. The process of claim 50, wherein the deferasirox or a pharmaceutically acceptable salt thereof has less than about 1400 parts per million methanol.

52. The process of claim 50, wherein the deferasirox or a pharmaceutically acceptable salt thereof has the overall level of organic volatile impurities in an amount of less than about 1500 parts per million.

53. A pharmaceutical composition comprising the highly pure deferasirox or a pharmaceutically acceptable salt thereof of claim 1 and one or more pharmaceutically acceptable excipients.

54. The pharmaceutical composition of claim 53, wherein the pharmaceutical composition is a solid dosage form, an oral suspension, a liquid, a powder, an elixir, an aerosol, syrups or an injectable solution.

55. The pharmaceutical composition of claim 53, wherein the highly pure deferasirox or a pharmaceutically acceptable salt thereof has a D₉₀ particle size of less than or equal to about 400 microns.

56. The pharmaceutical composition of claim 55, wherein the deferasirox or a pharmaceutically acceptable salt thereof has a D₉₀ particle size of less than or equal to about 300 microns, less than or equal to about 100 microns, less than or equal to about 60 microns, or less than or equal to about 15 microns.

57. (canceled)
